(19) United States
(12) Patent Application Publication
(10) Pub. No.: US 2026/0235788 A1
(43) Pub. Date: Aug. 13, 2026

(54) CONTACT LENS CONFIGURED TO RELEASE ACTIVE AGENT

(71) Applicant: Alcon Inc., Fribourg (CH)

(72) Inventors: Zahra Bassampour, Alpharetta, GA (US); Newton T. Samuel, Suwanee, GA (US); Lilanni Perez, Decatur, GA (US); Steve Yun Zhang, Sugar Hill, GA (US); Feng Jing, Alpharetta, GA (US); Claire Elizabeth Nason-Tomaszewski, Atlanta, GA (US); Jack W. Trieu, Atlanta, GA (US); Frank Chang, Cumming, GA (US); Troy Vernon Holland, Suwanee, GA (US); Adam K. Sniady, Lilburn, GA (US)

(21) Appl. No.: 19/537,817

(22) Filed: Feb. 12, 2026

Related U.S. Application Data
(60) Provisional application No. 63/757,952, filed on Feb. 13, 2025.

(51) Int. Cl. G02B 1/04; A61K 9/00; C08L 33/02

(52) U.S. Cl. CPC G02B 1/043; A61K 9/0048; C08L 33/02; C08L 2201/10; C08L 2203/02

(57) ABSTRACT

An anchor copolymer having repeating units from: (a) at least one arylboronic acid vinylic monomer and (b) at least one carboxyl vinylic monomer can be used to at or near a surface of a soft hydrogel contact lens and used to directly, or indirectly bind an active agent to the soft contact lens. In some aspects, a polyphenol can be bound to the anchor copolymer of the soft contact lens and a wetting agent bound to the polyphenol. Such a soft hydrogel contact lens can be used to manage dry eye, for example.

This application claims the benefits under 35 USC § 119(e) of U.S. provisional application No. 63/757,952, filed on 13 Feb. 2025, incorporated by reference in its entirety.

### TECHNICAL FIELD

The present disclosure relates to hydrogel contact lenses configured to release active agents. In particular, the present disclosure relates to a soft hydrogel contact lens composed of a hydrogel lens body and including an anchor copolymer, which can bind, directly or indirectly, an active agent.

### BACKGROUND

Controlled release of comfort agents or pharmaceutical agents (i.e. active agents) has long been a desired target for contact lenses but with limited success. Problems include insufficient, uncontrolled, or undesirable release profile as well as complex manufacturing or compromised optical properties of contact lenses including such active agents.

Hence, a continuing need exists for contact lenses configured to release active agents.

### SUMMARY OF THE DISCLOSURE

An advantage of the present disclosure is a soft hydrogel contact lens configured to release an active agent. Such contact lenses advantageously can be prepared with a variety of preformed soft contact lenses and in water-based processes.

These and other advantages are satisfied, at least in part, by a soft contact lens having a hydrogel lens body that includes an anchor copolymer and an active agent bound directly or indirectly to the anchor copolymer. Advantageously, the active agent can be a drug and/or comfort agent such as a wetting agent. The anchor copolymer includes: (a) repeating units of at least one arylboronic acid vinylic monomer, and (b) repeating units of at least one carboxyl vinylic monomer. In some aspects, the anchor copolymer includes (a) repeating units of the at least one arylboronic acid vinylic monomer from about 0.5% by mole to about 30% by mole, and (b) repeating units of the at least one carboxyl vinylic monomer from about 70% by mole to about 99.5% by mole. In one aspect, a mole percent of (a) and a mole percent of (b) sum to 100%. Other repeating units derived from other vinylic monomers can be included in the anchor copolymer, provided that the sum of the mole percentages of all repeating units including the mole percent of (a) and (b) sum to 100%.

In an implementation, an active agent can be delivered to an eye of a patient by applying to the eye of the patient the soft contact lens of the present disclosure. Advantageously, the method can include managing or treating an ocular disorder or disease of a patient in need thereof and delivering an effective amount of the active agent to manage or treat the ocular disorder or disease. For example, the method can include managing dry eye of a patient in need thereof and delivering an effective amount of a wetting agent as the active agent to manage the dry eye.

In another implementation, a soft contact lens having an active agent can be prepared by contacting a soft contact lens having a hydrogel lens body and an anchor polymer with an aqueous solution including an active agent to bind the active agent to the anchor copolymer. Alternatively, a soft contact lens can include a polyphenol, in which case the active agent can be bound to the polyphenol. For example, a soft contact lens having an active agent can be prepared by contacting a soft contact lens having a hydrogel lens body, an anchor polymer and a polyphenol bound to the anchor copolymer with an aqueous solution including an active agent to bind the active agent to the polyphenol. The soft contact lens can be contacted with various aqueous solutions of anchor copolymer, polyphenol and active agents by dipping or submersing the contact lens in the solution. The contact lens and/or solutions can have a temperature above room (e.g., above about 20° C.) such as from about 30° C. to about 140° C., e.g., from about 35° C. to about 90° C.

Additional aspects of the present disclosure include one or more of the following features individually or combined. For example, the hydrogel lens body can incorporate a polymeric material having 1,2- and/or 1,3-diol moieties and the anchor copolymer can be covalently attached to the hydrogel lens body through linkages each formed between one of the boronic acid groups of the anchor copolymer and one of the 1,2-diol and/or 1,3-diol moieties of the polymeric material. In other aspects, the hydrogel lens body can incorporate a polymeric material having first reactive functional groups at and/or near a surface thereof, the first reactive functional groups including carboxyl groups, amino groups, azetidnium groups, epoxide groups, aziridine groups, vinylsulfone groups, thiol groups, or any combination thereof; and one or more carboxyl and/or boronic acid groups of the anchor copolymer can react with one first reactive functional group at a temperature above 20° C. to form a covalent linkage. In further aspects, the active agent can be bound directly to one or more arylboronic acid groups and/or to one or more carboxyl groups of the anchor copolymer. In still further aspects, a polyphenol can be bound to the anchor copolymer and the active agent bound to the polyphenol.

Additional advantages of the present disclosure will become readily apparent to those skilled in the art from the following detailed description, wherein only certain aspects are shown and described, simply by way of illustration of carrying out certain subject matter. As will be realized, the invention is capable of other and different embodiments, and its several details are capable of modifications in various respects, all without departing from the invention. Accordingly, the drawings and description are to be regarded as illustrative in nature, and not as restrictive.

### BRIEF DESCRIPTION OF THE DRAWINGS

Reference is made to the attached drawings, wherein elements having the same reference numeral designations represent similar elements throughout and wherein:

FIG. 1 schematically illustrates a soft contact lens having an anchor copolymer composed of repeating units including pendent boronic acid and carboxyl groups in which a drug or comfort agent (e.g., lubricating agent) reversibly complexes with the pendent boronic acid group of the copolymer.

FIG. 2A schematically illustrates a soft contact lens having an anchor copolymer composed of repeating units including pendent boronic acid and carboxyl groups in which boronic acid groups are complexed with a 1,3-diol of PDMS bulk material in the contact lens body.

FIG. 2B schematically illustrates how a wetting agent, e.g., a polyamidoamine epichlorohydrin (PAE), can complex with the anchor copolymer such as the anchor copolymer incorporated in the soft contact lens of FIG. 2A.

FIG. 3A illustrates optical images of a silicone hydrogel contact lens having various coatings as provided in Example 1.

FIG. 3B illustrates an optical image of the silicone hydrogel contact lens of FIG. 3A after post rubbing and handling.

FIGS. 4A and 4B illustrate a schematic of a hydrogel contact lens having an active agent thereon though attachment via a polyphenol bound to an anchor copolymer in an implementation of the present disclosure.

### DETAILED DESCRIPTION OF THE DISCLOSURE

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any sub-combination.

Unless otherwise defined herein, scientific and technical terms used in connection with the present application have the meanings that are commonly understood by those of ordinary skill in the art to which this application pertains. Further, unless otherwise required by context, singular terms include pluralities and plural terms include the singular.

As used in the specification including the appended claims, the singular forms “a,” “an,” and “the” include the plural, and reference to a particular numerical value includes at least that particular value, unless the context clearly dictates otherwise.

As used in the specification including the appended claims, when a range of values is expressed, such range includes from the one particular value and/or to the other particular value. All ranges are inclusive and combinable. Further, reference to values stated in ranges includes each and every value within that range.

The term “about” as used herein when referring to a measurable value such as an amount, a temporal duration, and the like, is meant to encompass standard variations of the value as would be understood by those of ordinary skill in the art to which this application pertains as of its earliest filing date.

“Optional” or “optionally” means that the subsequently described event or circumstance can or cannot occur, and that the description includes instances where the event or circumstance occurs and instances where it does not.

An “ophthalmic device”, as used herein, refers to a contact lens (hard or soft), an intraocular lens, a corneal onlay, other ophthalmic devices (e.g., stents, glaucoma shunt, or the like) used on or about the eye or ocular vicinity.

“Contact Lens” refers to a device intended to be worn directly on an eye. A contact lens can be used to correct vision conditions, act as a therapeutic device, and/or act as a cosmetic.

A “hydrogel contact lens” refers to a contact lens comprising a hydrogel bulk (core) material. A hydrogel bulk material can be a non-silicone hydrogel material or a silicone hydrogel material.

A “hydrogel” or “hydrogel material” or “hydrogel lens body” refers to a crosslinked polymeric material which has three-dimensional polymer networks (i.e., polymer matrix), is insoluble in water, but can hold at least 10% by weight of water in its polymer matrix when it is fully hydrated (or equilibrated).

A siloxane, also referred to as a silicone, is an organosilicon group made up of alternating silicon and oxygen atoms. Siloxanes can be linear or cyclic, and usually have one or two organic groups attached to each silicon atom. The basic form of siloxane is one atom of oxygen linked to two atoms of silicon such as shown by: —Si—O—Si— where each Si atom includes one or two organic groups as substituents.

A “silicone hydrogel” or “SiHy” refers to a silicone-containing hydrogel obtained by copolymerization of a polymerizable composition comprising at least one silicone-containing monomer, or at least one silicone-containing macromer, or at least one crosslinkable silicone-containing prepolymer.

As used in this application, the term “non-silicone hydrogel” refers to a hydrogel that is theoretically free of silicon.

“Hydrophilic,” as used herein, describes a material or portion thereof that will more readily associate with water than with lipids. In contrast, and as used herein, “hydrophobic” describes a material or portion thereof that will more readily associate with lipids than with water.

The term “insoluble”, in reference to a compound or material in a solvent, means that the compound or material can be dissolved in the solvent to give a solution with a concentration of less than 0.01% by weight at room temperature (as defined above).

An “organic-base solution” refers to a solution that comprises at least 55% by weight of one or more organic solvent (i.e., that is formed by dissolving/blending a solute in an organic based solvent). It is understood that an organic based solution can comprise less than 45% by weight of water.

An “organic based solvent” refers to a solvent system comprising at least 55% by weight of one or more organic solvent.

An “aqueous solution” refers to a solution comprising at least 55% by weight of water. It is understood that an aqueous solution can comprise less than 45% by weight of one or more organic solvents miscible with water.

A “vinylic monomer” refers to a compound that has one sole ethylenically unsaturated group, and can be polymerized actinically or thermally.

The term “ethylenically unsaturated group” is employed herein in a broad sense and is intended to encompass any groups containing at least one >C═CH₂ group. Exemplary ethylenically unsaturated groups include without limitation (meth)acryloyl

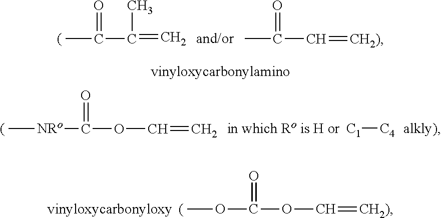

allyl, vinyl, styrenyl, or other C═C containing groups.

As used herein, “actinically” in reference to curing, crosslinking or polymerizing of a polymerizable composition, a prepolymer or a material means that the curing (e.g., crosslinked and/or polymerized) is performed by actinic irradiation, e.g., UV/visible light irradiation, or the like. Thermal curing or actinic curing methods are well-known to a person skilled in the art.

An “acrylic monomer” refers to a vinylic monomer having one sole (meth)acryloyl group. Examples of acrylic monomers includes (meth)acryloxy [or (meth)acryloyloxy] monomers and (meth)acrylamido monomers.

An “(meth)acryloxy monomer” or “(meth)acryloyloxy monomer” refers to a vinylic monomer having one sole group of

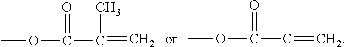

An “(meth)acrylamido monomer” refers to a vinylic monomer having one sole group of

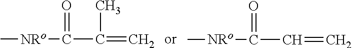

in which R^{o} is H or C₁-C₄ alkyl.

The terms “(C₁-C₄ alkyl) acryl . . . ” and “(meth)acryl . . . ” refer to acryl-based monomers with or without the (C₁-C₄ alkyl) or (meth) group, respectively. For example, the term “(meth)acrylic acid” refers to methacrylic acid and/or acrylic acid.

An “N-vinyl amide monomer” refers to an amide compound having a vinyl group (—CH═CH₂) that is directly attached to the nitrogen atom of the amide group.

The term “ene group” refers to a monovalent radical of CH₂═CH— or CH₂═CCH₃— that is not covalently attached to an oxygen or nitrogen atom or a carbonyl group.

An “ene monomer” refers to a vinylic monomer having one sole ene group.

A “vinyloxycarbonylamino monomer” refers to a vinylic monomer having one sole vinyloxycarbonylamino group.

A “vinylaminocarbonyloxy monomer” refers to a vinylic monomer having one sole vinylaminocarbonyloxy group.

A “vinylaminocarbonylamino monomer” refers to a vinylic monomer having one sole vinylaminocarbonylamino group.

A “hydrophilic vinylic monomer” refers to a vinylic monomer which, when polymerized alone, yields a homopolymer that is water-soluble or can absorb at least 10% by weight of water. However, it is understood that a hydrophilic vinylic monomer can be polymerized with other monomers in which the resulting copolymer may or may not be hydrophilic.

A “hydrophobic vinylic monomer” refers to a vinylic monomer which, when polymerized alone, yields a homopolymer that is insoluble in water and can absorb less than 10% by weight of water. However, it is understood that a hydrophobic vinylic monomer can be polymerized with other monomers in which the resulting copolymer may or may not be hydrophobic.

As used in this application, the term “vinylic crosslinker” refers to an organic compound having at least two ethylenically unsaturated groups. A “vinylic crosslinking agent” refers to a vinylic crosslinker having a molecular weight of 700 Daltons or less.

As used in this application, the term “polymer” means a material formed by polymerizing/crosslinking one or more monomers or macromers or prepolymers or combinations thereof.

A “macromer” or “prepolymer” refers to a compound or polymer that contains multiple ethylenically unsaturated groups and has a number average molecular weight of greater than 700 Daltons.

As used in this application, the term “molecular weight” of a polymeric material (including monomeric or macromeric materials) refers to the number-average molecular weight unless otherwise specifically noted or unless testing conditions indicate otherwise. A skilled person knows how to determine the molecular weight of a polymer according to known methods, e.g., GPC (gel permeation chromatography) with one or more of a refractive index detector, a low-angle laser light scattering detector, a multi-angle laser light scattering detector, a differential viscometry detector, a UV detector, and an infrared (IR) detector; MALDI-TOF MS (matrix-assisted laser desorption/ionization time-of-flight mass spectroscopy); ¹H NMR (Proton nuclear magnetic resonance) spectroscopy, etc.

A “polysiloxane segment” or “polydiorganosiloxane segment” interchangeably refers to a polymer chain segment (i.e., a divalent radical) of

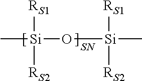

in which SN is an integer of 3 or larger and each of R_{S1} and R_{S2} independent of one another are selected from the group consisting of: C₁-C₁₀ alkyl; phenyl; C₁-C₄-alkyl-substituted phenyl; C₁-C₄-alkoxy-substituted phenyl; phenyl-C₁-C₆-alkyl; C₁-C₁₀ fluoroalkyl; C₁-C₁₀ fluoroether; aryl; aryl C₁-C₁₈ alkyl; -alk-(OC₂H₄)_{γ1}—OR^{o} (in which alk is C₁-C₆ alkylene diradical, R^{o} is H or C₁-C₄ alkyl and γ1 is an integer from 1 to 10); a C₂-C₄₀ organic radical having at least one functional group selected from the group consisting of hydroxyl group (—OH), carboxyl group (—COOH), amino group (—NR_{N1}R_{N1}′), amino linkages of —NR_{N1}—, amide linkages of —CONR_{N1}—, amide of —CONR_{N1}R_{N1}′, urethane linkages of —OCONH—, and C₁-C₄ alkoxy group, or a linear hydrophilic polymer chain, in which R_{N1} and R_{N1}′ independent of each other are hydrogen or a C₁-C₁₅ alkyl; and a photochromic organic radical having a photochromic group.

A “polydiorganosiloxane vinylic monomer” or “polysiloxane vinylic monomer” interchangeably refers to a compound comprising at least one polysiloxane segment and one sole ethylenically-unsaturated groups.

A “polydiorganosiloxane vinylic crosslinker” or “polysiloxane vinylic crosslinker” interchangeably refers to a compound comprising at least one polysiloxane segment and at least two ethylenically-unsaturated groups.

The term “fluid” as used herein indicates that a material is capable of flowing like a liquid.

As used in this application, the term “clear” in reference to a polymerizable composition means that the polymerizable composition is a transparent solution or liquid mixture having a light transmissibility of 85% or greater (preferably 90% or greater) in the range between 400 to 700 nm.

A free radical initiator can be either a photoinitiator or a thermal initiator. A “photoinitiator” refers to a chemical that initiates free radical crosslinking/polymerizing reaction by the use of light. A “thermal initiator” or “thermal free radical initiator” interchangeably refers to a chemical that initiates free radical crosslinking/polymerizing reaction by the use of heat energy.

The term “monovalent radical” and “monovalent group” interchangeably refer to an organic radical or group that is obtained by removing a hydrogen atom from an organic compound and that forms one bond with one other group in an organic compound. Examples include without limitation, alkyl (by removal of a hydrogen atom from an alkane), alkoxy (or alkoxyl) (by removal of one hydrogen atom from the hydroxyl group of an alkyl alcohol), thiyl (by removal of one hydrogen atom from the thiol group of an alkylthiol), cycloalkyl (by removal of a hydrogen atom from a cycloalkane), cycloheteroalkyl (by removal of a hydrogen atom from a cycloheteroalkane), aryl (by removal of a hydrogen atom from an aromatic ring of the aromatic hydrocarbon), heteroaryl (by removal of a hydrogen atom from any ring atom), amino (by removal of one hydrogel atom from an amine), etc.

The term “divalent radical” and “divalent group” interchangeably refer to an organic radical or group that is obtained by removing two hydrogen atoms from an organic compound and that forms two bonds with other two groups in an organic compound. For example, an alkylene divalent radical (i.e., alkylenyl) is obtained by removal of two hydrogen atoms from an alkane, a cycloalkylene divalent radical (i.e., cycloalkylenyl) is obtained by removal of two hydrogen atoms from the cyclic ring.

In this application, the term “substituted” in reference to an alkyl or an alkylenyl means that the alkyl or the alkylenyl comprises at least one substituent which replaces one hydrogen atom of the alkyl or the alkylenyl and is selected from the group consisting of hydroxyl (—OH), carboxyl (—COOH), —NH₂, sulfhydryl (—SH), C₁-C₄ alkyl, C₁-C₄ alkoxy, C₁-C₄ alkylthio (alkyl sulfide), C₁-C₄ acylamino, C₁-C₄ alkylamino, di-C₁-C₄ alkylamino, and combinations thereof.

The term “silicone hydrogel lens formulation” or “SiHy lens formulation” interchangeably refers to a polymerizable composition that comprises all necessary polymerizable components for producing a SiHy contact lens or a SiHy lens bulk material as well known to a skilled person.

A “UV-absorbing vinylic monomer” refers to a compound comprising one sole ethylenically-unsaturated group and can absorb predominantly UV light between 280 nm to 380 nm.

A “HEVL-absorbing vinylic monomer” refers to a compound comprising one sole ethylenically-unsaturated group and can absorb HEVL between 380 nm and 450 nm. It is understood that a HEVL-absorbing vinylic monomer can also absorb UV light between 280 nm and 380 nm.

In general, the present disclosure is directed to soft contact lenses having a hydrogel lens body and includes an anchor copolymer and an active agent bound directly or indirectly to the anchor copolymer. The anchor copolymer can be composed of: (a) repeating units of at least one arylboronic acid vinylic monomer, and (b) repeating units of at least one carboxyl vinylic monomer. Advantageously, the anchor copolymer provides reactive arylboronic acid and carboxyl groups that can facilitate anchoring the copolymer to the hydrogel lens body and/or binding an active agent to the soft contact lens. Active agents that can be used with soft contact lenses of the present disclosure include, for example, a comfort agent, such as a wetting agent, e.g., guar, e.g., hydroxypropyl-Guar, a hyaluronate, e.g., sodium hyaluronate, polyvinylalcohol (PVA), a polyethylene glycol, a propylene glycol; a carboxymethylcellullose, etc. a drug such as an antimuscarinic agent to manage myopia, a drug to treat ocular diseases such as dry eye, glaucoma, cataracts, allergic eyes, retinal tear, inflammation such as uveitis, a drug such as an antibiotic to treat an ophthalmic pathogen, a topical agent, etc. Such active agents can be delivered to the eye of a patient.

In some implementations, an active agent can be delivered to an eye of a patient by applying to the eye of the patient the soft contact lens of the present disclosure. The active agent can be delivered in an effective amount to manage or treat the ocular disorder or disease. For example, a method of delivering an active agent to an eye of a patient can include managing or treating an ocular disorder or disease of a patient in need thereof and delivering an effective amount of the active agent to manage or treat the ocular disorder or disease. For example, the method can include managing dry eye of a patient in need thereof and delivering an effective amount of a wetting agent as the active agent to manage the dry eye.

Advantageously, the anchor copolymer of the present disclosure is highly soluble in an aqueous medium and can be entangled or chemically bond to the hydrogel lens body of a soft contact lens. In some implementations, the anchor copolymer can be prepared from a combination of: (a) one or more arylboronic acid vinylic monomers, and (b) one or more carboxyl vinylic monomers. Polymerizing the monomers provide a copolymer having repeating units of the at least one arylboronic acid vinylic monomer and repeating units of the at least one carboxyl vinylic monomer.

### Arylboronic Acid Vinylic Monomer

Useful repeating units of an arylboronic acid vinylic monomer for the anchor copolymer can be derived from at least one phenylboronic acid vinylic monomer of formula (II):

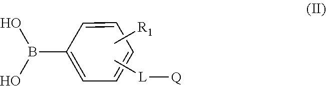

wherein R¹ is H, NO₂, F, Cl, or CF₃; Q is a monovalent radical of

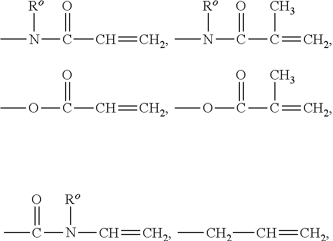

or —CH═CH₂; and L is a direct bond, a C₁-C₄ alkylene divalent radical, a divalent radical of

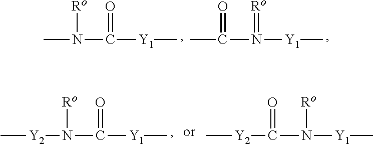

in which Y₁ is CH(OH), or a C₁-C₄ alkylene divalent radical, Y₂ is a C₁-C₄ alkylene divalent radical, and R⁰ is H or a C₁-C₄ alkyl.

Examples of arylboronic acid vinylic monomer of formula (II) include, without limitation: 3-vinylphenylboronic acid, 4-vinylboronic acid, 3-(meth)acrylamidophenylboronic acid, 4-(meth)acrylamidophenylboronic acid, or any combination thereof.

In addition, examples of arylboronic acid vinylic monomer of formula (II) can include a reaction product of an amino-containing phenylboronic acid derivative with (meth)acrylic acid halide, or a reaction product of an amino-containing phenylboronic acid derivative with a carboxy-containing vinylic monomer in the presence of a carbodiimide (e.g., 1-ethyl-3-(3-dimethylaminopropyl) carbodiimide (EDC), N,N′-dicyclohexylcarbodiimide (DCC), 1-cylcohexyl-3-(2-morpholinoethyl) carbodiimide, diisopropyl carbodiimide, or mixtures thereof) and N-hydroxysuccinimide, or a reaction product of a carboxy-containing phenylboronic acid derivative with an amino-containing vinylic monomer in the presence of a carbodiimide (e.g., 1-ethyl-3-(3-dimethylaminopropyl) carbodiimide (EDC), N,N′-dicyclohexylcarbodiimide (DCC), 1-cylcohexyl-3-(2-morpholinoethyl) carbodiimide, diisopropyl carbodiimide, or mixtures thereof) and N-hydroxysuccinimide, and combinations thereof.

Examples of preferred amino-containing phenylboronic acid derivatives include without limitation 3-aminophenylboronic acid, 4-aminophenylboronic acid, 4-amino-3-nitrophenylboronic acid, 4-amino-4-fluorophenylboronic acid, 2-(aminomethyl)-5-nitrophenylboronic acid, 3-(aminomethyl)-phenylboronic acid, 3-amino-5-nitrophenylboronic acid, 3-amino-3-(4-boronophenyl) propanoic acid, and combinations thereof.

Examples of preferred carboxy-containing vinylic monomer include without limitation 2-acrylamidoglycolic acid, 3-acrylamidopropionic acid, 4-acrylamidobutanoic acid, 5-acrylamidopentanoic acid, 3-acryloyloxypropanoic acid, 4-acryloyloxybutanoic acid, 5-acryloyloxypentanoic acid, and combinations thereof.

Examples of carboxy-containing phenylboronic acid derivatives include without limitation 3-carboxyphenylboronic acid, 4-carboxyphenylboronic acid, 3-boronophenylacetic acid, 4-boronophenylacetic acid, 2-(4-boronophenyl)-2-methylpropanoic acid, 3-(4-boronophenyl) propanoic acid, 3-(3-boronophenyl) propanoic acid, 5-(3-boronophenyl) pentanoic acid, 5-(4-boronophenyl) pentanoic acid, 4-(2-carboxyethyl)-3-nitrophenylboronic acid, 3-(3-carboxypropyonylamino)phenylboronic acid, 3-amino-3-(4-boronophenyl) propanoic acid, and combinations thereof.

Examples of amino-containing vinylic monomers include without limitation amino-C₂-C₄ alkyl (meth)acrylate, C₁-C₃ alkylamino-C₂-C₄ alkyl (meth)acrylate, amino-C₂-C₄ alkyl (meth)acrylamide, C₁-C₃ alkylamino-C₂-C₄ alkyl (meth)acrylamide, vinylamine, allylamine, and combinations thereof.

### Carboxyl Vinylic Monomer

A variety of carboxyl vinylic monomer can be used to prepare the repeating units of the anchor copolymer. As used in this application, the terms “carboxyl vinylic monomer” and “carboxyl-containing vinylic monomer” refer to any vinylic monomer having a carboxyl group (—COOH). Examples of carboxyl-containing vinylic monomers that can be used to prepare an anchor copolymer include, without limitation, a (C₁-C₄ alkyl) acrylic acid, such as (meth)acrylic acid, ethylacrylic acid, propylacrylic acid, a (C₁-C₄ alkyl) acrylamidoglycolic acid, such as 2-(meth)acrylamidoglycolic acid, 3-(meth)acrylamidopropionic acid, 4-(meth)acrylamidobutanoic acid, 5-(meth)acrylamidopentanoic acid, 3-(meth)acryloyloxypropanoic acid, 4-(meth)acryloyloxybutanoic acid, 5-(meth)acryloyloxypentanoic acid, 2-carboxyethyl (meth)acrylate, or any combination thereof.

In some implementations, the anchor copolymer includes: (a) from about 0.5% by mole to about 30% by mole, such as from about 1% to about 25% by mole, e.g., from about 2% to about 20% by mole, or from about 3% to about 15% by mole of repeating units of the at least one arylboronic acid vinylic monomer, and (b) from about 70% by mole to about 99.5% by mole, such as from about 75% to about 99% by mole, e.g., from about 80% to about 98% by mole, or from about 85% to about 97% by mole of repeating units of the at least one carboxyl vinylic monomer. In one aspect, a mole percent of (a) and a mole percent of (b) sum to 100%. Other repeating units derived from other vinylic monomers can be included in the anchor copolymer, provided that the sum of the mole percentages of all repeating units including the mole percent of (a) and (b) sum to 100%.

Forming the Base Coating with the Anchor Copolymer

Advantageously, the anchor copolymer can be located at or near the surface of the soft contact lens and anchored to the hydrogel lens body. Further, the active agent can be bound directly or indirectly to the anchor copolymer, e.g., the active agent can be bound directly to one or more arylboronic acid groups and/or to one or more carboxyl groups of the anchor copolymer. In still further aspects, a polyphenol can be bound to the anchor copolymer and the active agent bound to the polyphenol.

There are a variety of ways that the anchor polymer can be incorporated into the hydrogel lens body. Generally, processes of preparing a soft hydrogel contact lens of the present disclosure can include contacting, e.g., dipping or submerging, a soft contact lens having a hydrogel lens body with an aqueous solution of the anchor copolymer. A polyphenol can be bound to the anchor copolymer and/or the active agent can be bound to the anchor copolymer or polyphenol by contacting a soft contact lens having a hydrogel lens body, an anchor polymer with an aqueous solution of the polyphenol or an aqueous solution of the active agent. The soft contact lens can be contacted with various aqueous solutions of anchor copolymer, polyphenol or active agents by dipping or submersing the contact lens in the solution. The contact lens and/or solutions can have a temperature above room (e.g., above about 20° C.) such as from about 30° C. to about 140° C., e.g., from about 35° C. to about 90° C.

In one implementation, the anchor copolymer can be physically entangled with the hydrogel lens body by contacting the soft contact lens with an aqueous solution of the anchor copolymer for a time sufficient to allow the anchor copolymer to penetrate the hydrogel lens body. For example, an anchor copolymer of the present disclosure can be dissolved in water (purified water, such as distilled water or deionized water). The pH of the aqueous solution can be from about 0 to about 3.5, e.g., from a pH of about 1.0 to about 2.5 and adjusted by adding an acid, such as hydrochloric acid, formic acid, acetic acid, malic acid, citric acid, nitric acid, phosphoric acid, sulfuric acid, etc. The concentration of the anchor copolymer in an aqueous solution can be from about 0.005% by weight to about 10%, such as from about 0.1% by weight to about 5% by weight, e.g., from about 0.02% by weight to about 2% by weight.

Further, the solution or hydrogel contact lens or both can have a temperature above room (e.g., above about 20° C.) such as from about 30° C. to about 140° C., e.g., from about 35° C. to about 90° C., or from about 40° C. to about 80° C. when contacting the soft contact lens with the anchor copolymer solution. Contacting the soft contact lens with the anchor copolymer solution can involve simply dipping the contact lens in a single bath of the solution for a period of time or alternatively dipping the contact lens sequentially in a series of baths of the solutions for a shorter time periods for each bath. In some aspects, the contact lens can be contacted with the aqueous solution of the anchor copolymer for a total time of up to about 240 minutes such as from about 60 minutes to about 240 minutes. After contacting the hydrogel contact lens with the solution, the contact lens can be rinsed with water or with a buffered saline solution, such as a phosphate buffered saline, having a pH from about 6.5 to about 9.5, such as from about 6.8 to about 8.0, e.g., from about 7.0 to about 7.5.

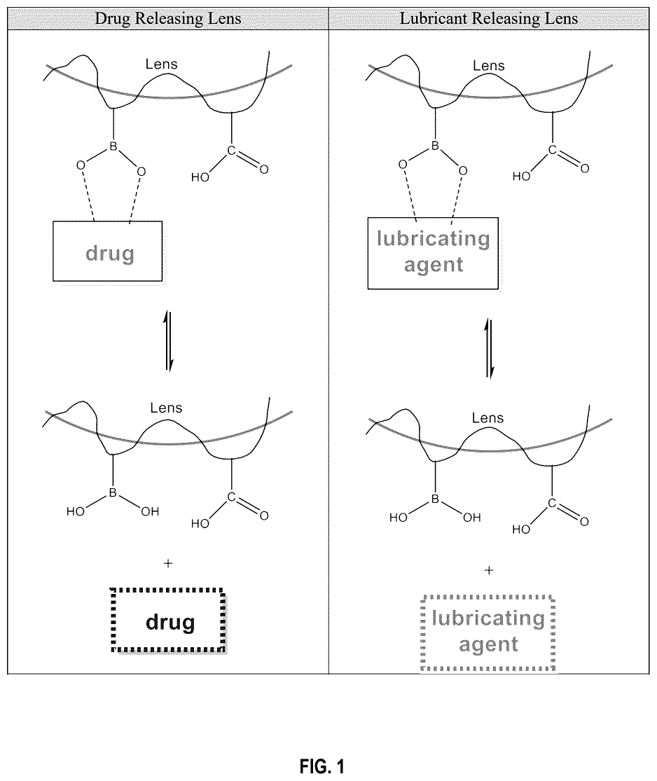

FIG. 1 schematically illustrates a soft contact lens having an anchor copolymer composed of repeating units that include boronic acid and carboxyl groups. As shown, the anchor copolymer can be located, at least in part, at or near a surface of the lens and secured in place by entangling the chain of the anchor copolymer with the hydrogel lens body of the soft contact lens. In addition, part of the anchor copolymer at or near the surface of the contact lens has pendent boronic acid and carboxyl groups available for reversable binding with an active agent, e.g., in the example of FIG. 1, a drug or lubricating agent. That is, in this example, the boronic acid moiety can undergo reversible/pH dependent complexation with 1,2- and 1,3-diols. (see also Scheme I below). This complexation enables uptake of active agents that can be released over time on eye. The uptake and release can be controlled by the pKa of the arylboronic acid vinylic monomer. Further, the uptake and release profile can be controlled, in part, by the mole percent of arylboronic acid groups in the anchor copolymer.

In another implementation, the anchor copolymer can be chemically bound to the hydrogel lens body as well as entangled with the hydrogel lens body. In this implementation, the soft contact lens comprises a hydrogel lens body, which comprises or is made of a polymeric material having 1,2- and/or 1,3-diol moieties (e.g., a crosslinked polyvinylalcohol or a polymeric material comprising diol-containing repeating units each having a 1,2- or 1,3-diol). Such a hydrogel contact lens can be coated with an anchor copolymer of the present disclosure by reaction between pendant boronic acid groups and 1,2-diol or 1,3-diol moieties in the hydrogel lens body to form reversible covalent bonds (i.e., cyclic boronic ester linkage), as illustrated in Scheme I below.

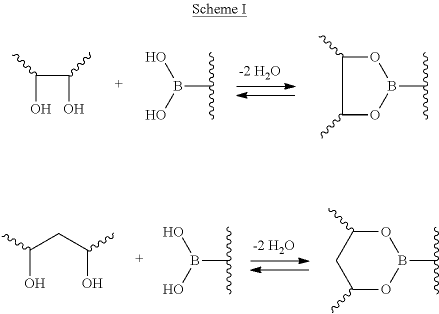

As shown in Scheme I above, when the hydrogel lens body incorporates a polymeric material having 1,2- and/or 1,3-diol moieties, the anchor copolymer can be covalently attached to the hydrogel lens body through linkages each formed between one of the boronic acid groups of the anchor copolymer and/or one of the 1,2-diol and/or 1,3-diol moieties of the polymeric material.

Advantageously, the anchor copolymer can be formed on the surface of a hydrogel contact lens incorporating a polymeric material having 1,2- and/or 1,3-diol moieties by a water-based process. For example, the formed cyclic boronic ester linkages can be stable in a sealed and autoclaved lens package in the pH range of a typical lens packaging solution. Resultant hydrogel contact lenses with an anchor copolymer thereon can enhance the surface lubricity of the hydrogel contact lens. Further, the water-based coating process can have minimal impact on the properties of the preformed hydrogel contact lens so as to maintain beneficial attributes of softness, equilibrium water content, elongation, oxygen permeability, etc.

A variety of soft contact lenses having a hydrogel lens body that incorporates a polymeric material having 1,2- and/or 1,3-diol moieties can be used with the anchor copolymer of the present disclosure. In one implementation, a preformed hydrogel contact lens can comprise or is made of a polymeric material that includes at least one type of diol-containing repeating unit in which each repeating unit has a 1,2- or 1,3-diol moiety. In some aspects such a polymeric material can have at least 5%, such as at least 10%, 15%, even at least 20% repeating-unit-equivalent of 1,2- and 1,3-diol moieties. It is understood that diol-containing repeating units can be derived, in a polymerization, either from at least one diol-containing vinylic monomer having at least one 1,2-diol and/or 1,3-diol moiety or from at least one diol-containing vinylic crosslinker having at least one 1,2-diol and/or 1,3-diol moiety. An anchor copolymer of the present disclosure can be bound to a soft contact lenses having a hydrogel lens body that incorporates a polymeric material having 1,2- and/or 1,3-diol moieties by immersing the contact lens in an aqueous solution of the anchor copolymer with heat to a temperature above room temperature, e.g., above about 20° C. such as from about 30° C. to about 140° C.

Any 1,2-diol and/or 1,3-diol containing vinylic monomers can be used to prepare a polymeric material. Examples of include without limitation N-2,3-dihydroxypropyl (meth)acrylamide, N-tris(hydroxymethyl) methyl(meth)acrylamide, 2-gluconamidoethyl (meth) acrylamide, 3-gluconamidopropyl (meth)acrylamide, glycerol (meth)acrylate, glucosyl (meth)acrylate, glucosyloxyethyl (meth)acrylate, sugar acrylic monomers disclosed in U.S. Pat. Appl. Pub. No. 2012/0264214 and U.S. Pat. No. 4,721,760 and the like, 3-allyoxy-1,2-propanediol, 2-allyloxymethyl-2-(hydroxymethyl)-1,3-propanediol, 2-allyloxymethyl-2-ethyl-1,3-propanediol (i.e., trimethylolpropaneallylether), allyl alpha-D-mamiopyranoside, allyl alpha-D-galactopyranoside, allyl 6-deoxyhexopyranoside, allyl 6-deoxy-2-O-methylhexopyranoside, and any combination thereof.

In addition, or alternatively, any 1,2-diol and/or 1,3-diol containing vinylic crosslinkers can be used to prepare a polymeric material. Examples include without limitations N,N′-(1,2-dihydroxyethylene) bis-(meth)acrylamide, N,N′ (2,3-dihydroxybutylene) bis-(meth)acrylamide, polydimethylsiloxane vinylic crosslinkers having diol-containing substituent as disclosed in U.S. Pat. Appl. Pub. No. 2017/01666673, and any combination thereof.

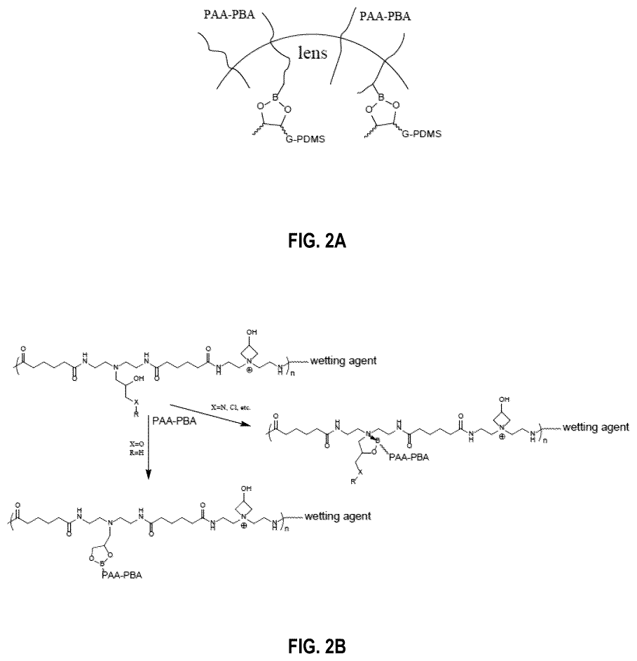

FIG. 2A schematically illustrates a soft contact lens having an anchor copolymer composed of repeating units that include pendent boronic acid and carboxyl groups, e.g. a polyacrylic acid-co-phenylboronic acid vinylic monomer (PAA-PBA). In addition to the chain entanglement mechanism of securing the anchor layer onto a soft lens surface, an anchor copolymer of the present disclosure can form boronic acid ester dynamic bonds with the hydrogel lens body which incorporates a polymeric material having 1,2- and/or 1,3-diol moieties. Such boronic acid ester dynamic bonds reinforces and strengthens the adherence of the anchor copolymer to the soft contact lens. For example, dynamic bonds can be formed between the anchor's boronic acid groups and the polymeric material's 1,2-diol or 1,3-diol groups. Such 1,2-diol or 1,3-diol groups can be incorporated in the polymeric material by copolymerizing macromers or monomers that include such groups, e.g., a glycerol-containing crosslinker, such as a glycerol-containing PDMS (G-PDMS), a glycerol-containing monomer, such as glycerol methacrylate. In the example of FIG. 2A, the boronic acid groups of the anchor copolymer complex with a 1,3-diol of G-PDMS bulk material in the contact lens body.

In addition, and as shown in FIG. 2A, part of the anchor copolymer at or near the surface of the contact lens has pendent boronic acid and carboxyl groups available for reversable binding with an active agent. In the example of FIG. 2B, a comfort agent, e.g., wetting agent such as a polyamidoamine epichlorohydrin (PAE), can complex with the anchor copolymer. This binding enables uptake of the active agent that can be released over time on eye. The uptake and release can be controlled by the pKa of the arylboronic acid vinylic monomer. Further, the uptake and release profile can be controlled, in part, by the mole percent of arylboronic acid groups in the anchor copolymer.

In another implementation, the soft contact lens can include a hydrogel lens body that incorporates a polymeric material having first reactive functional groups at and/or near a surface thereof. Such first reactive functional groups can include carboxyl groups, amino groups, azetidnium groups, epoxide groups, aziridine groups, vinylsulfone groups, thiol groups, or any combination thereof. Such first reactive functional groups are capable of reacting with one or more carboxyl and/or boronic acid groups of the anchor copolymer. In some aspects, one or more first reactive functional groups of the polymeric material can react with one or more carboxyl and/or boronic acid groups of the anchor copolymer at a temperature above 20° C. to form a covalent linkage. For example, an anchor copolymer of the present disclosure can be bound to a soft contact lenses having a hydrogel lens body that incorporates first reactive groups by immersing the contact lens in a solution of the anchor copolymer with heat to a temperature above room temperature, e.g., from about 30° C. to about 140° C.

In some implementations, the active agent can be bound directly to one or more arylboronic acid groups of the anchor copolymer. In other implementations, the active agent is bound directly to one or more carboxyl groups of the anchor copolymer. In still further implementations, the active agent can be bound indirectly to the anchor copolymer.

### Polyphenol Layer

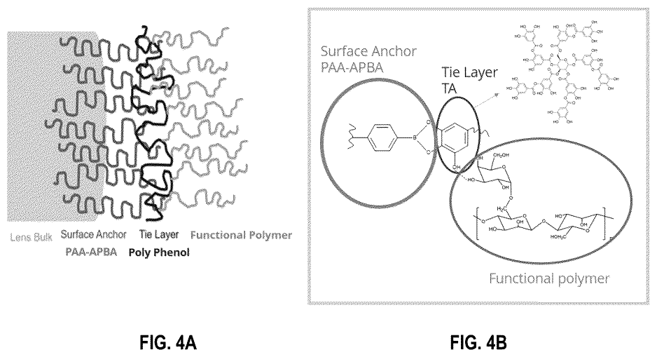

Advantageously, anchor copolymers of the present disclosure readily bind to polyphenols which in turn can act as an intermediate layer on the contact lens and further facilitate reversible bonding of an active agent to the contact lens. A polyphenol (also known as a polyhydroxyphenol) as used herein refers to a compound with at least three phenol groups in which each phenol group has one or more exposed hydroxyl groups. Preferably, polyphenols formed on the surface of a soft hydrogel contact lens have more than three phenol groups with each phenol having at least one exposed hydroxyl groups. Example of polyphenols useful in the present disclosure include plant-derived polyphenols such as tannic acid, epigallocatechin gallate, epicatechin gallate, epigallocatechin, raspberry ellagitannin, theaflavin-3-gallate, tellimagrandin II, etc. or any combination thereof. A polyphenol can be bound to an anchor copolymer on a soft contact lens by immersing such a contact lens in an aqueous solution of the polyphenol. In addition, the contact lens or solution can be heating to a temperature above room temperature (above 20° C.), e.g., from about 30° C. to about 140° C. FIG. 4A, described further in Example 1, schematically illustrates a hydrogel contact lens having an active agent (functional polymer such as a wetting agent) thereon though attachment via a polyphenol bound to an anchor copolymer. As shown in FIG. 4A, a hydrogel lens body (lens bulk) can include an anchor copolymer that extends, in part, from the hydrogel lens body to a surface of the contact lens. A polyphenol can be bound to the anchor copolymer, acting as a tie layer, and an active agent bound to the polyphenol. The examples below further describe such a soft contact lens.

### EXAMPLES

The following examples are intended to further illustrate certain aspects of the subject technology and are not limiting in nature. Those skilled in the art will recognize, or be able to ascertain, using no more than routine experimentation, numerous equivalents to the specific substances and procedures described herein.

### Finger Lubricity Test.

The lubricity of a contact lens is evaluated by using finger-felt lubricity test which characterizes qualitatively the slipperiness of a lens surface on a friction rating scale from 0 to 4. The higher the friction rating is, the lower the slipperiness (or lubricity). Commercial lenses: DAILIES TOTAL1®; ACUVUE® OASYS™; ACUVUE ADVANCE PLUS™; DAILIES Aqua Comfort Plus® and AIR OPTIX®, are assigned a friction rating (designated “FL” hereinafter) of 0, 1, 2, 3, and 4 respectively. They are used as standard lenses for determining the friction rating of a lens under test. The samples are place in PBS for at least two rinses of 30 minutes each and then transferred to fresh PBS before the evaluation. Before the evaluation, hands are rinsed with a soap solution, extensively rinsed with DI water and then dried with KimWipe® towels. The samples are handled between the fingers and a numerical number is assigned for each sample relative to the above standard lenses described above.

### Sudan Black Stain Test

A Sudan Black staining test involves dipping a contact lenses into a Sudan Black dye solution (Sudan Black in the mixture −80% mineral oil and −20% vitamin E oil). Sudan Black dye is hydrophobic and has a great tendency to be adsorbed by a hydrophobic material or onto a hydrophobic lens surface or hydrophobic spots on a partially coated surface of a hydrophobic lens (e.g., silicone hydrogel contact lens). If the coating on a hydrophobic lens is intact, no staining spots should be observed on or in the lens. All of the lenses under test are fully hydrated. Visible fine lines on lens surface may indicate the presence of cracking of the crosslinked coatings.

### Example 1

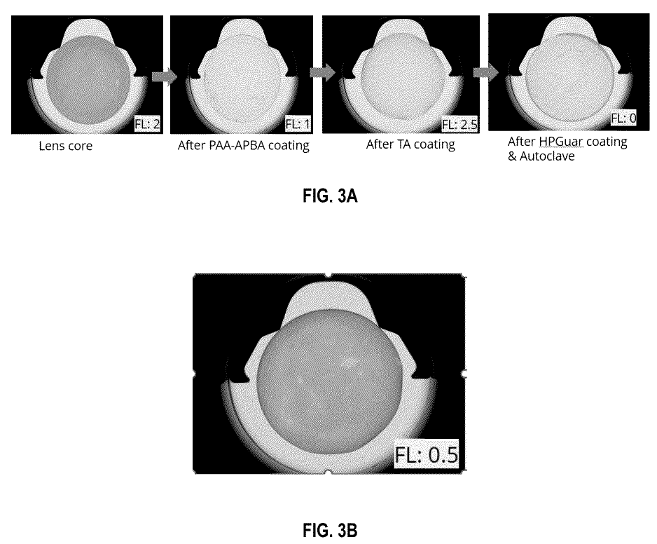

An anchor copolymer including 90 mole % of acrylic acid and 10 mole % of 3-acrylamido phenylboronic acid (PAA-APBA) (90-10 mol %) was dissolved in deionized water and the pH lowered to less than about 2 using formic acid to prepare a 0.1% aqueous solution of PAA-APBA (90-10 mol %). Preformed silicone hydrogel contact lenses were submerged in the solution (0.1% of PAA-APBA (90-10 mol %)) for about 120 minutes at about 40° C. The contact lenses were then rinsed for about 25 minutes in deionized water (DI-H₂O) at room temperature and then introduced to a 1% tannic acid solution for about 30 min at about 40° C. The contact lenses were then rinsed in DI-H2O for about 30 min at room temperature and were placed in blister packages containing about 1000 ppm hydroxypropyl-guar (HPGuar) solution. The blister packages were subjected to autoclave conditions of 45 min at 121° C. After certain of the above steps, the silicone hydrogel contact lenses were evaluated for Sudan Black (SB) staining and Finger Lubricity (FL). The results of these tests are provided in Table 1 below and shown in FIG. 3A.

TABLE 1

| | Sudan Black | Finger |
|---|---|---|
| Process Step | (SB) Test | Lubricity Test |
| Initial Preformed Hydrogel | Strong staining | 2 |
| Contact Lens (No coating) | | |
| After PAA-APBA coating | Colorless | 1 |
| After TA coating | Colorless | 2.5 |
| After HPGuar coating | Colorless | 0 |

As provided in Table 1 and shown in FIG. 3A, the preformed, uncoated hydrogel contact lens exhibited an FL of 2 and was heavily stained by the SB test. These results indicate the initial surface of the silicone hydrogel contact lens was hydrophobic and not very lubricious. After subjecting the lens to the PAA-APBA solution, the silicon hydrogel lens exhibited a reduced FL to 1 and the lens was colorless and clear. These results are consistent with the PAA-APBA anchor copolymer on the surface of the contact lens and forming a coating layer on the surface since the entire surface lacked visible staining. After subjecting the PAA-APBA coated lens to the tannic acid solution, the FL increased to 2.5 suggesting incorporation of the tannic acid groups. The lens was still clear after SB staining, which indicated a TA coating layer on the lens. After subjecting the TA coated lens to the HPGuar solution and autoclaving, the FL dropped to zero and the lens remained clear and colorless after SB testing. The SB and FL data show that a PAA-APBA copolymer can be anchored on a prefabricated contact lens and further that a polyphenol (e.g., tannic acid) can be attached to the anchor and a comfort agent (e.g., HGuar) can be attached to the polyphenol.

### Release of Comfort Agent

The silicone hydrogel contact lenses prepared with the PAA-APBA anchor copolymer, polyphenol bound thereto and comfort agent HGuar bound to the polyphenol as described above were subjected to mechanical rubbing. Briefly, the lenses out-of-pack were rinsed in PBS saline for at least 30 min and left in fresh PBS. Before manual rubbing of lenses, hands are rinsed with a soap solution, extensively rinsed with DI water and then dried with KimWipe® towels. The lens is placed in the palm of one hand and rubbed with the index finger of other hand for 1 minute with some PBS saline added to keep everything hydrated. The lens is inverted and again rubbed for 1 minute on the other side. The lenses subjected to such rubbing were then tested for SB staining. FIG. 3B shows an optical image of the silicone hydrogel contact lens after post rubbing and handling. As shown in FIG. 3B, the lens was stained by SB. The change to a SB stained lens from a colorless lens prepared with a comfort agent shows the comfort agent was released from the lens with mechanical rubbing. The lens, however, was still somewhat lubricious likely due to some of the PAA-APBA anchor copolymer and potentially polyphenol tannic acid remaining on the lens surface.

FIGS. 4A and 4B schematically illustrate a mechanism that the comfort agent is believed bound to the contact lens is the Example above. As shown in FIG. 4A, the anchor copolymer is believed entangled with the bulk polymeric material of the hydrogel contact lens. Certain of the anchor copolymer extends to the surface where carboxyl and boronic acid groups are available to react with the polyphenol (tannic acid). For this example, the tannic acid effectively acts as a tie layer between the anchor layer (PAA-APBA) and the comfort agent (HPGuar). The polyols on the tannic acid can complex with the borates present in anchor layer (PAA-APBA) and also form H-bonds with HPGuar (functional polymer in FIGS. 4A and 4B). Once the soft contact lens having the comfort agent is placed on eye, glucose and salt in the tear film will compete with the diol-borate and H-bond interactions, thereby releasing the comfort agent from the contact lens. In addition, the repeated movement of the eyelid over the lens surface also promotes release of the beneficial agent attached to the coating through the reversible cross-links. Such soft contact lenses can be used for controlled release of a comfort agent, e.g., to help relieve a patient with dry eye symptoms.

Only certain features and aspects of the subject technology and examples of its versatility are shown and described in the present disclosure. It is to be understood that the technology disclosed herein is capable of use in various other combinations and environments and is capable of changes or modifications. Thus, for example, those skilled in the art will recognize, or be able to ascertain, using no more than routine experimentation, numerous equivalents to the specific substances, procedures and arrangements described herein. Such equivalents are considered to be within the scope of the invention and are covered by the following claims.

What is claimed is:

1. A soft contact lens, comprising
a hydrogel lens body including an anchor copolymer and an active agent bound directly or indirectly to the anchor copolymer;
wherein the anchor copolymer comprises: (a) repeating units of at least one arylboronic acid vinylic monomer and (b) repeating units of at least one carboxyl vinylic monomer.

2. The soft contact lens of claim 1, wherein the hydrogel lens body incorporates a polymeric material having 1,2- and/or 1,3-diol moieties; and wherein the anchor copolymer is covalently attached to the hydrogel lens body through linkages each formed between one of the boronic acid groups of the anchor copolymer and one of the 1,2-diol and/or 1,3-diol moieties of the polymeric material.

3. The soft contact lens of claim 1, wherein the hydrogel lens body incorporates a polymeric material having first reactive functional groups at and/or near a surface thereof, the first reactive functional groups including carboxyl groups, amino groups, azetidnium groups, epoxide groups, aziridine groups, vinylsulfone groups, thiol groups, or any combination thereof; and wherein one or more carboxyl and/or boronic acid groups of the anchor copolymer is capable of reacting with one first reactive functional group at a temperature above 20° C. to form a covalent linkage.

4. The soft contact lens of claim 1, wherein the active agent is bound directly to one or more arylboronic acid groups of the anchor copolymer.

5. The soft contact lens of claim 1, wherein the active agent is bound directly to one or more carboxyl groups of the anchor copolymer.

6. The soft contact lens of claim 1, further comprising a polyphenol bound to the anchor copolymer and the active agent bound to the polyphenol.

7. The soft contact lens of claim 1, wherein the active agent is a drug or comfort agent.

8. The soft contact lens of claim 1, wherein repeating units of the at least one carboxyl vinylic monomer is derived from (meth)acrylic acid, ethylacrylic acid, propylacrylic acid, 2-(meth)acrylamidoglycolic acid, 3-(meth)acrylamidopropionic acid, 4-(meth)acrylamidobutanoic acid, 5-(meth)acrylamidopentanoic acid, 3-(meth)acryloyloxypropanoic acid, 4-(meth)acryloyloxybutanoic acid, 5-(meth)acryloyloxypentanoic acid, or any combination thereof.

9. The soft contact lens of claim 8, wherein repeating units of the at least one arylboronic acid vinylic monomer is derived from at least one phenylboronic acid vinylic monomer of formula (II): 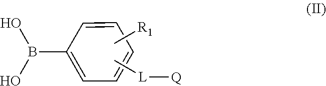
wherein R₁ is H, NO₂, F, Cl, or CF₃; Q is a monovalent radical of 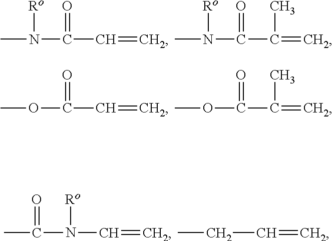
or —CH═CH₂; and L is a direct bond, a C₁-C₄ alkylene divalent radical, a divalent radical of 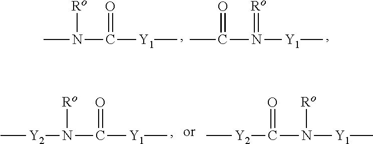
in which Y₁ is CH(OH), or a C₁-C₄ alkylene divalent radical, Y₂ is a C₁-C₄ alkylene divalent radical, and R⁰ is H or a C₁-C₄ alkyl.

10. The soft contact lens of claim 9, wherein repeating units of the at least one arylboronic acid vinylic monomer is derived from 3-vinylphenylboronic acid, 4-vinylphenylboronic acid, 3-(meth)acrylamidophenylboronic acid, 4-(meth)acrylamidophenylboronic acid, or any combination thereof.

11. The soft contact lens of claim 9, wherein the anchor copolymer comprises: (a) from about 0.5% by mole to about 30% by mole of repeating units of the at least one arylboronic acid vinylic monomer, and (b) from about 70% by mole to about 99.5% by mole of repeating units of the at least one carboxyl vinylic monomer, provided a mole percent of (a) and a mole percent of (b) sum to 100%.

12. A method of delivering an active agent to an eye of a patient, the method comprising:
applying to the eye of the patient the soft contact lens to deliver an active agent to the eye of the patient, wherein the soft contact lens comprises a hydrogel lens body including an anchor copolymer and the active agent bound directly or indirectly to the anchor copolymer, wherein the anchor copolymer comprises: (a) repeating units of at least one arylboronic acid vinylic monomer and (b) repeating units of at least one carboxyl vinylic monomer.

13. The method of claim 12, wherein the method comprises managing or treating an ocular disorder or disease of a patient in need thereof and delivering an effective amount of the active agent to manage or treat the ocular disorder or disease.

14. The method of claim 13, wherein the method comprises managing dry eye of a patient in need thereof and delivering an effective amount of a wetting agent as the active agent to manage the dry eye.

15. A process of preparing a soft contact lens configured to release an active agent, according to Procedure (1) or (2):
wherein Procedure (1) comprises a step of contacting a soft contact lens having a hydrogel lens body and an anchor polymer with an aqueous solution including an active agent to bind the active agent to the anchor copolymer, wherein the anchor copolymer comprises: (a) repeating units of at least one arylboronic acid vinylic monomer and (b) repeating units of at least one carboxyl vinylic monomer;
wherein Procedure (2) comprises a step of contacting a soft contact lens having a hydrogel lens body, an anchor polymer and a polyphenol bound to the anchor copolymer with an aqueous solution including an active agent to bind the active agent to the polyphenol, wherein the anchor copolymer comprises: (a) repeating units of at least one arylboronic acid vinylic monomer and (b) repeating units of at least one carboxyl vinylic monomer.

16. The process of claim 15, wherein Procedure (1) or (2) further comprises a step of contacting a soft contact lens having a hydrogel lens body with a solution of an anchor polymer to form the soft contact lens having the hydrogel lens body and the anchor polymer.

17. The process of claim 16, wherein Procedure (2) further comprises a step of contacting a soft contact lens having a hydrogel lens body and an anchor polymer with a solution of a polyphenol to form the soft contact lens having the hydrogel lens body, the anchor polymer and the polyphenol bound to the anchor copolymer.
